(19) United States
(12) Patent Application Publication
(10) Pub. No.: US 2008/0004845 A1
(43) Pub. Date: Jan. 3, 2008

(54) Method and system for the calculation of dose responses for radiotherapy treatment planning

(76) Inventors: Gregory Alexander Failla, Gig Harbor, WA (US); Todd Arlin Wareing, Gig Harbor, WA (US); John Morton McGhee, Hollis, NH (US); Douglas Allen Barnett, Pattersonville, NY (US)

Correspondence Address: OLYMPIC PATENT WORKS PLLC, P.O. BOX 4277, SEATTLE, WA, 98104, US

(21) Appl. No.: 11/809,774

(22) Filed: Jun. 1, 2007

Related U.S. Application Data
(63) Continuation-in-part of application No. 11/726,386, filed on Mar. 21, 2007.
Application No. 11/726,386 is a continuation-in-part of application No. 11/499,862, filed on Aug. 3, 2006.
Application No. 11/499,862 is a continuation-in-part of application No. 11/273,596, filed on Nov. 14, 2005.
Application No. 11/273,596 is a continuation-in-part of application No. 10/910,239, filed on Aug. 2, 2004, now abandoned.
Application No. 10/910,239 is a continuation-in-part of application No. 10/801,506, filed on Mar. 15, 2004, now abandoned.
(60) Provisional application No. 60/454,768, filed on Mar. 14, 2003.
(60) Provisional application No. 60/491,135, filed on Jul. 30, 2003.
(60) Provisional application No. 60/505,643, filed on Sep. 24, 2003.

(51) Int. Cl. A61B 5/00

(52) U.S. Cl. 703/2

(57) ABSTRACT

Method and system that allows patient dose response functions to be calculated, prior to treatment planning, for external photon beam radiotherapy. In one embodiment, for each location where a separate dose value is desired, referred to as a dose region, adjoint calculations are performed to calculate the adjoint solution fields associated with that dose region. Once potential beam directions are specified, a ray tracing process is performed to transport the adjoint solution fields out of the patient and to locations where treatment plan parameters may be specified. The output of this process is the dose response at each dose region resulting from a prescribed photon fluence, at a given location, direction, and energy.

### CROSS-REFERENCE TO RELATED APPLICATIONS

This application is a continuation-in-part of application Ser. No. 11/726,386, filed Mar. 21, 2007, which is a continuation-in-part of application Ser. No. 11/499,862, filed Aug. 3, 2006, which is a continuation-in-part of application Ser. No. 11/273,596, filed Nov. 14, 2005, which is a continuation-in-part of application Ser. No. 10/910,239, filed Aug. 2, 2004, which is a continuation-in-part of application Ser. No. 10/801,506, filed Mar. 15, 2004, which claims the benefit of provisional Application Nos. 60/454,768, filed Mar. 14, 2003; 60/491,135, filed Jul. 30, 2003; and 60/505,643, filed Sep. 24, 2003

### TECHNICAL FIELD

The present invention relates to computer simulations of radiation transport, and in particular, the application of such simulations to radiotherapy dose calculations.

### BACKGROUND OF THE INVENTION

Clinical Need

In external photon beam radiotherapy treatments, such as intensity modulated radiotherapy (IMRT) and stereotactic radiosurgery (SRS), a clinical need exists for real time dose calculations during treatment planning. IMRT and SRS treatment plans are generally developed using inverse planning techniques, where dozens of dose calculations may be performed to best satisfy objective functions and to develop an optimized patient specific plan. Treatment plans are generally optimized real-time, with a physicist sitting at the computer. To achieve clinically viable times for treatment plan optimization, therefore, dose calculations must be performed almost real-time. Because of this, most clinical treatment planning systems in use today employ simple dose calculations methods, which are fast but inaccurate, during treatment plan optimization. While more accurate methods such as Monte Carlo may be used for final dose verification, time constraints generally prohibit their effective use during treatment plan optimization.

A typical course of radiotherapy may involve twenty or more treatments, referred to as fractions, given over a period of several weeks. In traditional photon beam radiotherapies, a patient specific treatment plan is developed prior to the first treatment, and this same treatment is given at every fraction. With the advent of image guided radiotherapy (IGRT), patients may be imaged prior to, or during each treatment fraction. Through real-time imaging, the potential exists to optimize treatment plans at every fraction, based on anatomical changes due to tumor response, breathing cycles, or other factors. Since patients are generally imaged in IGRT just prior to each treatment fraction, treatment plans must be optimized within a few minutes, requiring almost real time dose calculation speed. In all external beam modalities, treatment planning in a clinical framework is generally not possible except with the use simplified dose calculation methods, which are known to provide insufficient accuracy in many cases.

A compelling need exists for a dose calculation method which is both accurate and fast enough for clinical treatment planning.

Description of Photon Beam Radiotherapy

In external photon beam radiotherapy, a photon source 101 may be produced through a number of methods such as an electron beam impinging on a target in a linear accelerator. This photon source may then be collimated through field shaping devices such as the primary collimator 102, flattening filter 103, blocks 104, and multi-leaf collimators 105, to create a beam 106 having a desired spatial distribution which is delivered to an anatomical region 107. The radiation beam may be delivered through a rotating gantry, which will generally move to multiple locations 201 in the course of a single treatment. By delivering beams 202 from multiple locations 201 into the patient 203, the highest dose can be concentrated on the treatment region 204. At each gantry position, the field shaping devices are positioned to create a beam with the desired shape. In more advanced modalities, such as intensity modulated radiation therapy (IMRT), multi-leaf collimators may be used to deliver numerous fields at each gantry position, or alternatively produce a continuously varying field profile. By doing this, spatial variations in the beam intensity can be realized, leading to greater dose conformity.

As a photon passes into the anatomical region, a variety of particle interactions may occur, such as scattering, absorption, and secondary particle creation. As an example, a photon 301 passes into the anatomical region 302, and a collision occurs 303 which scatters the photon so that it has a new direction of travel and lower energy 304. The collision also produces a free electron that deposits its energy locally 305. The photon goes on to have another collision 306 where an electron is produced that deposits energy locally 307. The twice scattered photon 308 then exits the anatomical region at a lower energy.

In photon beam radiotherapies, photon energies are generally high enough so that secondary electrons, those produced by photon interactions inside the patient, may travel significant distances. Thus, it becomes necessary for dose calculation methods to accurately account for spatial electron transport. A majority of the dose in photon beam radiotherapies result from electrons produced by the first photon interactions inside the patient, referred to herein as primary photon interactions, and a much smaller percentage results from electrons produced by secondary photon interactions, or photons produced or scattered by a primary photon interaction.

Description of Treatment Planning Process

Prior to treatment planning, a radiation oncologist will generally identify and contour acquired image data (CT, MRI, etc.) to specify regions such as the planning treatment volume (PTV), organs-at-risk (OAR), and other critical structures. Additional points where the dose is of interest, or where optimization constraints are to be applied, may also be specified. The radiation oncologist may also specify desired minimum and maximum doses or other region specific parameters, which are used as objectives to drive the treatment planning process.

In traditional 3D conformal radiotherapy (3D-CRT), treatment planning is relatively straightforward. For each gantry position, generally only a single beam is delivered. The perimeter of beam will generally be similar to that of the PTV as viewed from that beam direction, and the field intensity is generally uniform throughout each beam. Once the gantry positions, or beam directions, are specified, in 3D-CRT often only the weight of each beam may be modified during the optimization process, over which there may generally be 3 to 9 beams in a treatment. Although the use of wedges may provide some spatial variations within each beam, treatment planning is still relatively straightforward.

In IMRT, dozens of beams, also referred to as fields, are delivered from each gantry position, where each field can have a different shape and duration. In dynamic IMRT, continuously variable field shapes may be delivered. IMRT can enable complex spatial distributions to be prescribed at each gantry position, increasing the potential for dose conformity, and the number of variables to be solved for in the optimization process. Inverse planning is generally performed, where desired dose objectives are prescribed in each physician contoured structure. Multiple optimization iterations are then needed to develop a patient specific treatment plan which best satisfies the dose objectives, with dose calculations performed in each iteration step.

For IMRT treatment planning, fields at each gantry position are commonly represented as fluence maps 401, which are generally 2-D grids oriented normally to the beam direction at each gantry position. Fluence maps describe the angular distribution of the time integrated photon source, and possibly electron source, delivered from each gantry position. The location of a grid element 501 in a fluence map 502 can be defined by a unique (k,l) index. Within each grid element, a spatially invariant photon fluence and spectrum is prescribed. All photons associated with a specific fluence map are assumed to originate from a single source point location. A planar beam may be represented by a source point far away from the fluence map, and a converging beam may be represented by a source point on the other side of the fluence map. Given that all photons are assumed to originate at the source point, or travel to the focal point in the case of a converging beam, the direction of a photon passing through any point in the fluence map is implicitly known. Separate fluence maps are sometimes used to model primary and scatter components. For example, the primary component exiting a treatment head may be represented by a fluence map associated with a point source located at the accelerator target, and the scatter component exiting the treatment head may be represented by a separate fluence map having a point source located below the flattening filter. Other variations exist. A single fluence map may be used to represent the integrated sum of all fields delivered from a given gantry position, rather than having a separate fluence map for each field. At each optimization step during treatment planning, the photon intensity and spectrum in each fluence map grid element may be independently modified. A dose calculation is then performed, using the prescribed fluence map(s) as input, to compute the patient dose distribution.

In SRS, the treatment may be delivered through dozens or hundreds of narrow beams, each from a different position. As such, each beam may be represented as having a fixed spatial distribution, or alternatively as a single, spatially invariant, photon intensity and spectrum. At each optimization step during treatment planning, the relative intensity of each beam, and the beam locations, may be modified. Other variations exist.

### SUMMARY OF THE INVENTION

Method embodiments of the present invention pre-calculate the dose response in a patient prior to treatment planning, enabling rapid dose calculations to be performed during treatment plan optimization. The process is based on an application of the solution to the adjoint forms of the Linear Boltzmann Transport Equation for neutral particles and the Linear Boltzmann Fokker-Plank Transport Equation for charged particles. The process can be applied to pre-calculate the dose response at any number of selected points, regions, or voxels.

In one embodiment of the present invention, the process involves first specifying locations where the dose is desired, referred to as dose regions. For each dose region, adjoint coupled electron and adjoint photon transport calculations are performed to compute the volumetric adjoint electron and adjoint photon flux fields, respectively, associated with that dose region. These solutions are stored, either in memory or on disk, for later use. Once potential beam locations are specified, ray tracing is performed to compute the first scattered photon and electron sources inside the patient anatomy. The adjoint flux solutions, output from the adjoint calculations, are then combined with the first scattered sources, output from the ray tracing process, to compute the dose response at each dose region. For each dose region, the dose response can be stored as a function of the prescribed photon angle, intensity and energy, for each of the beam positions from which ray tracing was performed.

There are many embodiments of the above process, which uses ray tracing to transport adjoint calculated dose responses out of the patient, to locations where treatment plan parameters may be specified. The method is directly applicable to any photon beam radiotherapy modality, including 3D-CRT, IMRT, and SRS, and could also be adapted to support other treatments, such as electron beam and proton beam radiotherapies.

### BRIEF DESCRIPTION OF THE DRAWINGS

FIG. 1 illustrates a radiotherapy beam passing through field shaping devices of a linear accelerator, and into the patient anatomy.

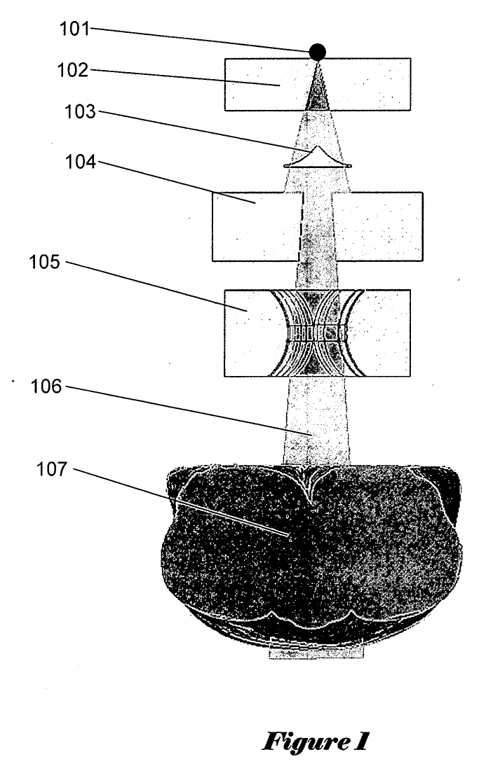

FIG. 2 illustrates a radiotherapy treatment where multiple external beams are delivered to the patient anatomy, which converge on the treatment volume.

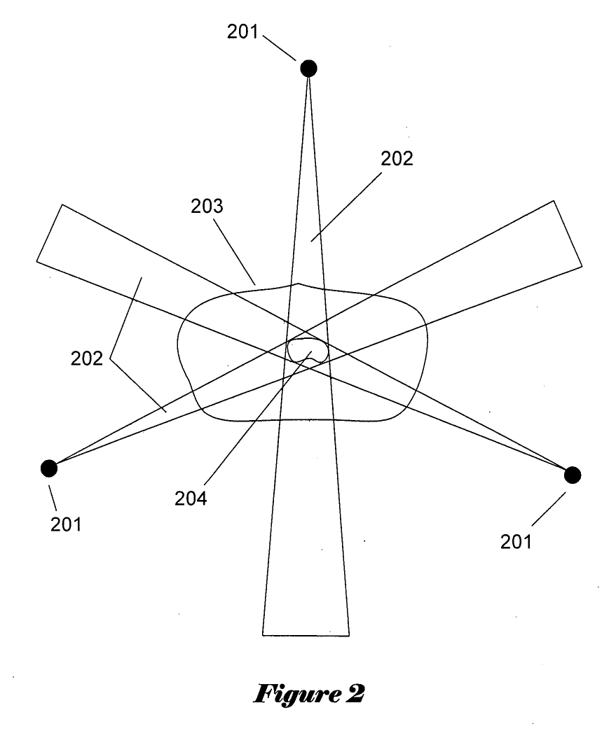

FIG. 3 illustrates relevant photon and electron interactions in the patient anatomy for external photon beam radiotherapy.

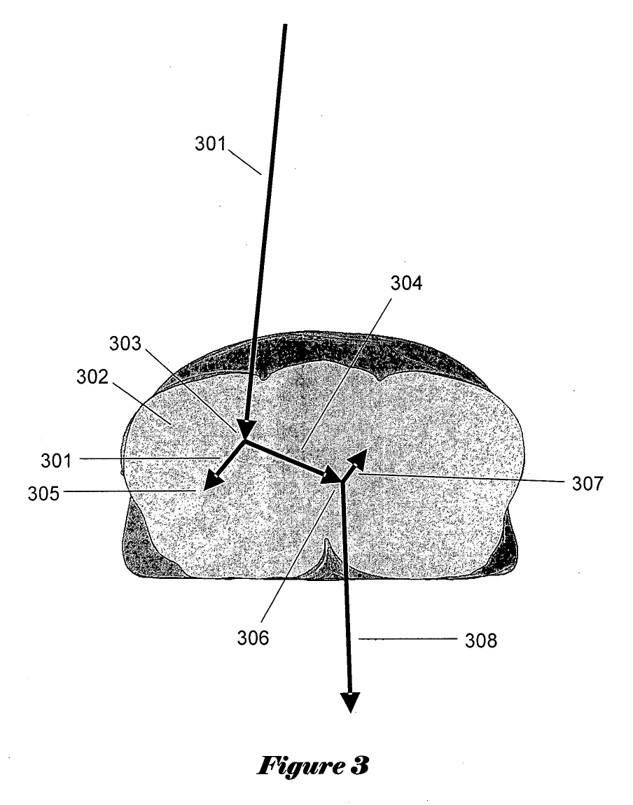

FIG. 4 illustrates a radiotherapy treatment with multiple beams, where fluence maps are used to describe the spatial and angular distribution of photons delivered from each beam.

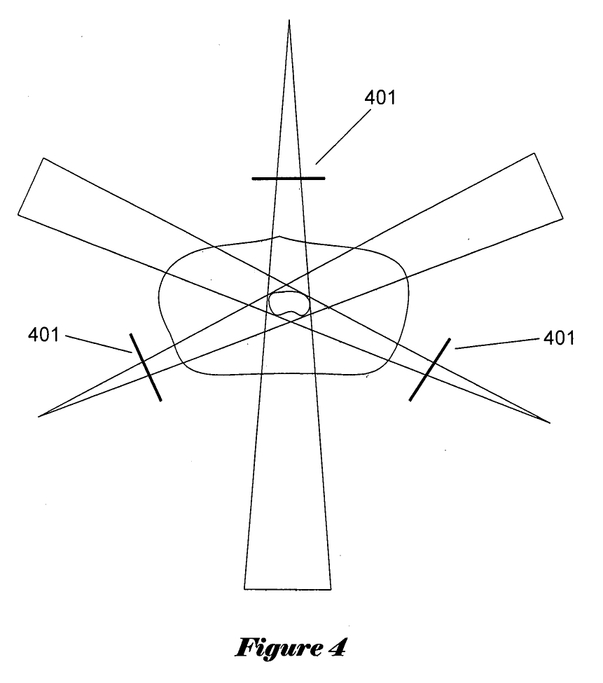

FIG. 5 illustrates photons passing through a fluence map, comprising discrete grid elements defined by (k,l) locations.

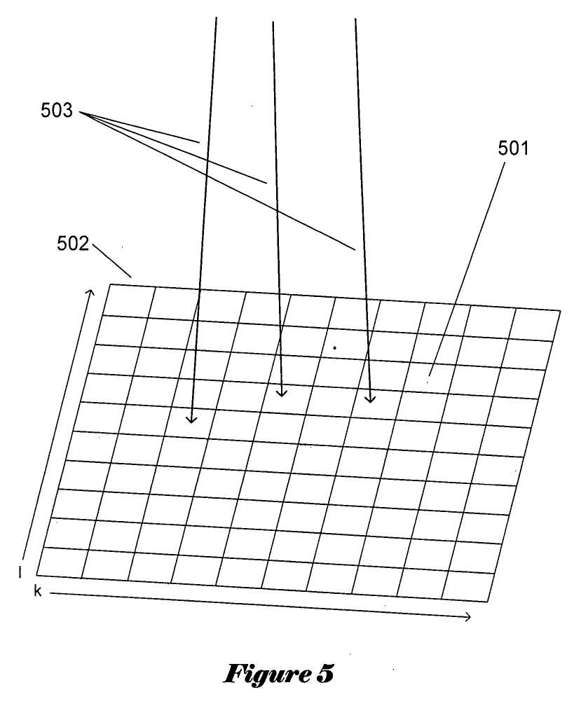

FIG. 6 illustrates a slice of a three dimensional computational grid constructed over a patient volume.

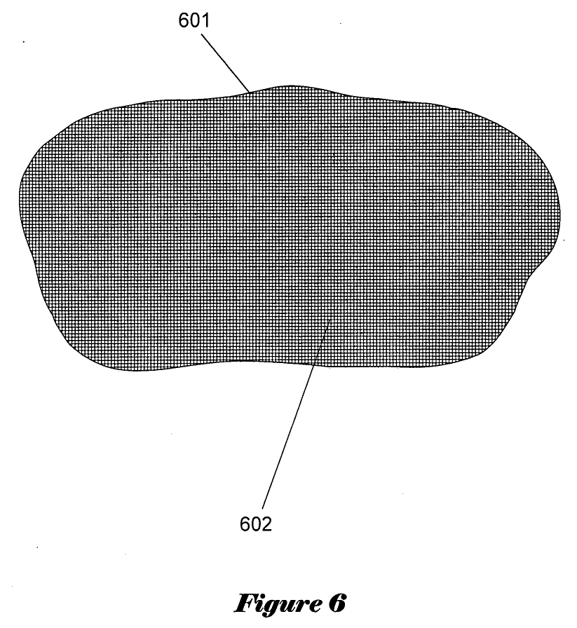

FIG. 7 illustrates a slice of a subset of three dimensional computational elements which are contained entirely inside, or which overlap, regions of interest. These elements comprise locations where the dose response is desired.

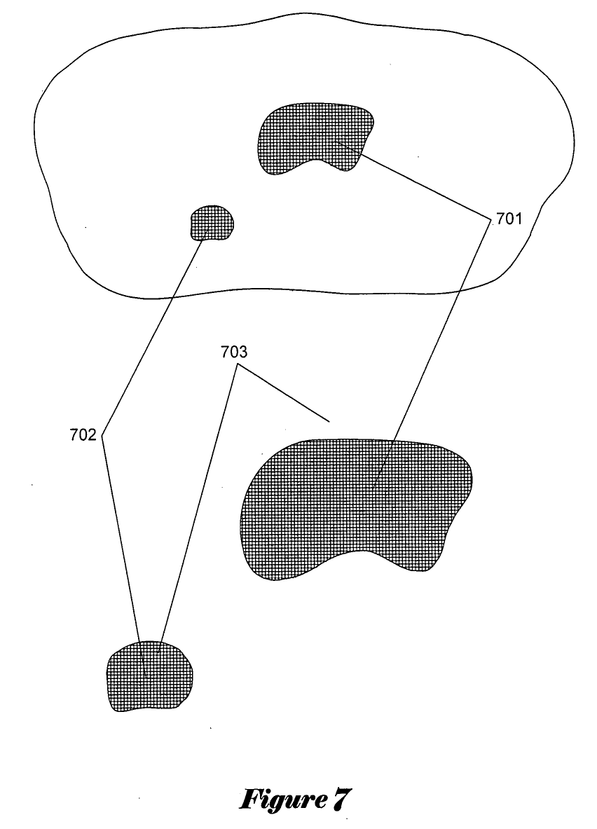

FIG. 8 illustrates a slice through a three dimensional computational grid on which an electron adjoint calculation is to be performed, which is a subset of the three dimensional grid constructed over the full patient volume, and which surrounds the element where the adjoint electron source is prescribed.

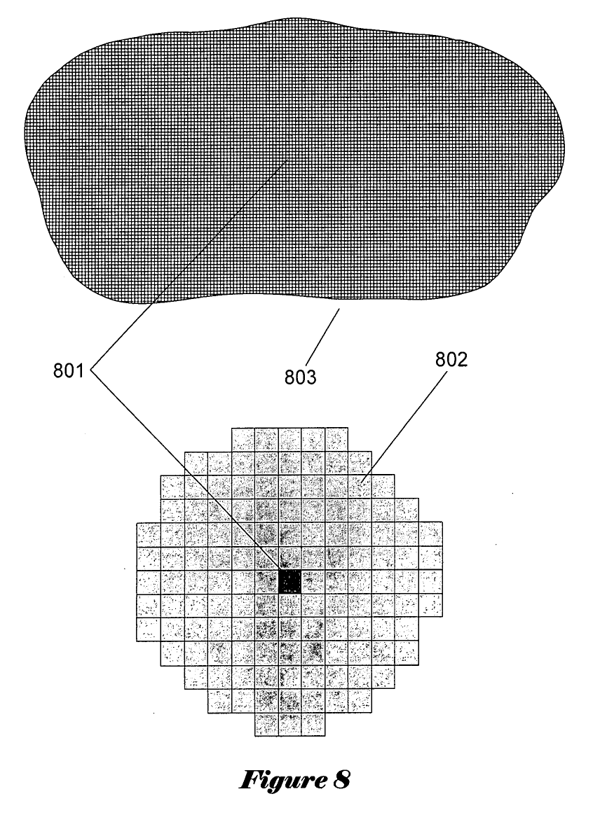

FIG. 9 illustrates a slice through a three dimensional computational grid on which a photon adjoint calculation is to be performed, which is a subset of the three dimensional grid constructed over the full patient volume, and which surrounds the element where the adjoint electron source is prescribed.

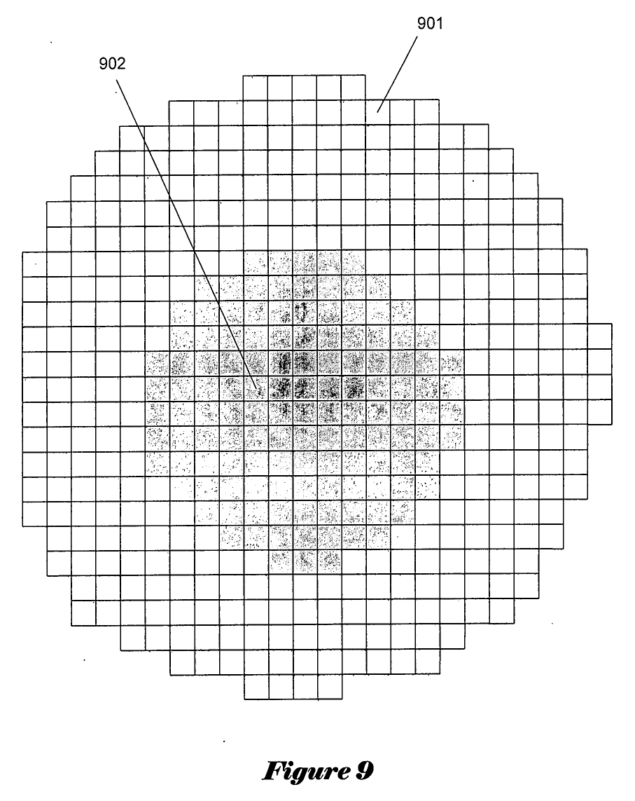

FIG. 10 illustrates the process of ray tracing to compute the first scattered photon and first scattered electron sources at voxels in the dose response grid, where each ray passes through a single (k,l) element of the fluence map associated with that source.

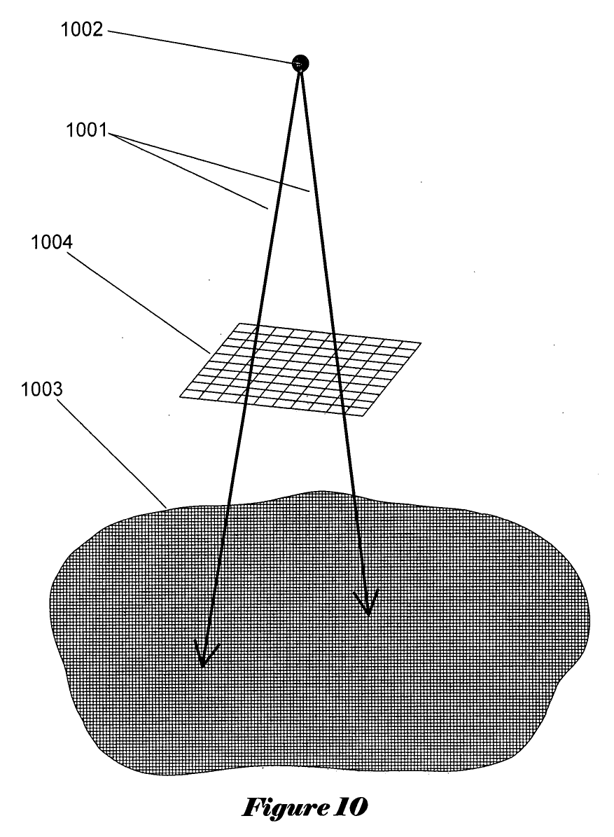

### DETAILED DESCRIPTION OF THE INVENTION

A method is presented for accurately pre-calculating the dose response at one or more points, voxels, or regions for the purposes of external photon beam radiotherapy treatment planning.

Following is a description of the governing forward and adjoint transport equations for neutral and charged particles, which is provided as an introduction. Since the immediate application is photon beam radiotherapy, only the coupling of electrons and photons is presented, though this does not preclude the use of other paired particle or single particle calculations.

Forward Coupled Photon-Electron Transport Equations

For a problem spatial domain with volume, V, and surface, δV, the time-independent, three-dimensional system of coupled Boltzmann transport equations (LBTE) to be solved are given by (for brevity the dependent variables have been suppressed in the equations):

$$\begin{matrix}\hat{Ω} \cdot {∇}^{->} {Ψ}^{γ} + {σ}_{t}^{γ} {Ψ}^{γ} = {q}^{γγ} + {q}^{γ} , & ( 1 a ) \\ \hat{Ω} \cdot {∇}^{->} {Ψ}^{e} + {σ}_{t}^{e} {Ψ}^{e} - \frac{\partial }{\partial E} {S}_{R} {Ψ}_{e} = {q}^{ee} + {q}^{γ e} + {q}^{e} , & ( 1 b )\end{matrix}$$

where Equations (1a) and (1b) solve for photon, γ, and electron, e, transport, respectively. Equations (1) are subject to all possible standard boundary conditions on δV, the most likely being the vacuum or non-reentrant boundary conditions given by:
Ψ^{γ}=0, for \hat{Ω}·\vec{n}<0, (2a)
Ψ^{e}=0, for \hat{Ω}·\vec{n}<0, (2b)
Here, Ψ^{γ} (\vec{r},E,\hat{Ω}) is the photon angular flux, Ψ^{e} (\vec{r},E,e) is the electron angular flux, \vec{r}=(x,y,z) is the spatial position vector, E is energy, \hat{Ω}=(μ,η,ξ) is the unit direction vector, and \vec{n} is the outward directed unit normal vector to surface δv. For Equations (1), \vec{r}∈V, \hat{Ω}∈4π, and E>0. The first term on the left hand side of Equations (1) is termed the streaming operator. The second term on the left hand side of Equations (1) is termed the collision or removal operator, where σₜ^{γ}(\vec{r},E) and σₜ^{e}(\vec{r},E) are the macroscopic photon and electron total cross sections, respectively. Equation (1b) is the Boltzmann Fokker-Plank transport equation, where the third term on the left represents the continuous slowing down (CSD) operator, where S_{R}(\vec{r},E) is the restricted collisional plus radiative stopping power, as defined in CEPXS [Ref. 1]. The right hand side of Equations (1) includes the scattering, production, and extraneous source terms (q^{γ} and q^{e}) The scattering and production sources are defined by:

$$\begin{matrix}{q}^{γγ} ( {r}^{->} , E , \hat{Ω} ) = {\int }_{0}^{∞} ⅆ {E}^{′} {\int }_{4 π} ⅆ {Ω}^{′} {σ}_{s}^{γγ} ( {r}^{->} , {E}^{′} -> E , \hat{Ω} \cdot {\hat{Ω}}^{′} ) {Ψ}^{γ} ( {r}^{->} , {E}^{′} , {\hat{Ω}}^{′} ) , & ( 3 a ) \\ {q}^{γ e} ( {r}^{->} , E , \hat{Ω} ) = {\int }_{0}^{∞} ⅆ {E}^{′} {\int }_{4 π} ⅆ {Ω}^{′} {σ}_{s}^{γ e} ( {r}^{->} , {E}^{′} -> E , \hat{Ω} \cdot {\hat{Ω}}^{′} ) {Ψ}^{γ} ( {r}^{->} , {E}^{′} , {\hat{Ω}}^{′} ) , & ( 3 b ) \\ {q}^{ee} ( {r}^{->} , E , \hat{Ω} ) = {\int }_{0}^{∞} ⅆ {E}^{′} {\int }_{4 π} ⅆ {Ω}^{′} {σ}_{s}^{ee} ( {r}^{->} , {E}^{′} -> E , \hat{Ω} \cdot {\hat{Ω}}^{′} ) {Ψ}^{e} ( {r}^{->} , {E}^{′} , {\hat{Ω}}^{′} ) , & ( 3 c )\end{matrix}$$

where, q^{γγ} is the photon source resulting from photon interactions, q^{γe} is the electron source resulting from photon interactions, and q^{ee} is the electron source resulting from electron interactions. σ_{S}^{γγ} is the macroscopic photon-to-photon differential scattering cross section, σ_{S}^{γe} is the macroscopic photon-to-electron differential production cross section, and σ_{S}^{ee} is the macroscopic electron-to-electron differential scattering cross section.

In the current description, only the coupling of electrons and photons is presented, however, this does not preclude the use of other paired particle or single particle calculations.

The basic assumptions used in Equations (1) come from the CEPXS cross sections [Ref. 1], and are briefly summarized as follows. Both charged pair secondary particles are assumed to be electrons instead of one electron and one positron. Also, the partial coupling technique of CEPXS is used, whereby photons produce electrons, but electrons do not produce photons. The energy from photons produced by the electrons is assumed to be deposited locally. Both these assumptions have only a minor effect on the energy deposition field. A primary assumption of Equation (1b) is that the Fokker-Plank operator (of which the CSD operator is the first order term), is used for “soft” interactions that results in small-energy losses. Catastrophic interactions that results in large energy losses are represented with the standard Boltzmann scattering. Higher order terms in the Fokker-Plank operator are not necessary for photon beam problems, but may be used when necessary for other coupled charged particle calculations.

The macroscopic differential scattering cross section is usually expanded into Legendre polynomials, Pₗ(μ₀), where μ₀=\hat{Ω}·\hat{Ω}′. This expansion allows the differential scattering or production cross section(s) to be expressed as:

$${σ}_{s}^{γγ / γ e / ee} ( {r}^{->} , {E}^{′} -> E , \hat{Ω} , {\hat{Ω}}^{′} ) = {\sum }_{l = 0}^{∞} \frac{2 l + 1}{4 π} {σ}_{s , l}^{γγ / γ e / ee} ( {r}^{->} , {E}^{′} -> E ) {P}_{l} ( {μ}_{0} ) . \qquad ( 4 )$$

Similarly, the angular flux appearing in the scattering source is expanded into spherical harmonics moments:

$$Ψ ( {r}^{->} , {E}^{′} , {\hat{Ω}}^{′} ) = {\sum }_{l = 0}^{∞} {\sum }_{m = - l}^{l} {ϕ}_{l , m} ( {r}^{->} , {E}^{′} ) {Y}_{l , m} ( {\hat{Ω}}^{′} ) , \qquad ( 5 )$$

where Y_{l,m}(\hat{Ω}) are the spherical harmonic functions and φ_{l,m}(\vec{r},E′) are the spherical harmonic moments of the angular flux given by

$${ϕ}_{l , m} ( {r}^{->} , E ) = {\int }_{4 π} ⅆ {Ω}^{′} {Y}_{l , m}^{*} Ψ ( {r}^{->} , {\hat{Ω}}^{′} , E ) . \qquad ( 6 )$$

* denotes the complex conjugate. Equations (4) through (6) are exact. A limit is generally set on the scattering order, 0≦l≦L, and hence the number of spherical harmonic moments kept in the scattering/production sources. Using the Legendre addition theorem, the scattering and production sources become:

$${q}^{γγ / γ e / ee} ( {r}^{->} , E , \hat{Ω} ) = {\sum }_{l = 0}^{L} {\sum }_{m = - l}^{l} {\int }_{0}^{∞} ⅆ {E}^{′} {σ}_{s , l}^{γγ / γ e / ee} ( {r}^{->} , {E}^{′} -> E ) {ϕ}_{l , m} ( {r}^{->} , {E}^{′} ) {Y}_{l , m} ( \hat{Ω} ) . \qquad ( 7 )$$

The upper limit, L, is chosen to accurately represent the anisotropy of the scattering source.

Discretization in space, angle, and energy is needed to solve Equations (1). There are numerous deterministic solution methods which may be used to solve Equations (1), some of which are described in [Ref. 2].

Once the electron angular flux is solved, the dose in any region, i, of the problem may be obtained through the following:

$${D}_{i} = {\int }_{0}^{∞} ⅆ E {\int }_{4 π}^{} ⅆ \hat{Ω} {\int }_{{V}_{vox}} ⅆ V \frac{{σ}_{ED}^{e} ( {r}^{->} , E )}{ρ} {Ψ}^{e} ( {r}^{->} , E , \hat{Ω} ) . \qquad ( 8 )$$

Here, σ_{ED}^{e}, is the macroscopic energy deposition cross sections in units of MeV/cm and ρ is the material density.
Photon Point Sources

In photon beam radiotherapy, the incident photon source(s) on the patient may be represented as one or more point sources, typically located at the treatment head target and possibly at other locations to represent photon scatter, such as below the flattening filter. Each source may have full distributions in both energy and angle, for which source anisotropy may be described by a fluence map. Such sources allow for specialized analytic methods to be used to transport the prescribed photon source into the patient anatomy.

For a photon point source, q^{γ}(E,\hat{Ω}), located at position, \vec{r}ₚ, Equations (1) become:

$$\begin{matrix}\hat{Ω} \cdot {∇}^{->} {Ψ}^{γ} + {σ}_{t}^{γ} {Ψ}^{γ} = {q}^{γγ} + {q}^{γ} ( E , \hat{Ω} ) δ ( {r}^{->} - {{r}^{->}}_{p} ) , & ( 9 a ) \\ \hat{Ω} \cdot {∇}^{->} {Ψ}^{e} + {σ}_{l}^{e} {Ψ}^{e} - \frac{\partial }{\partial E} {S}_{R} {Ψ}^{e} = {q}^{ee} + {q}^{γ e} + {q}^{e} , & ( 9 b )\end{matrix}$$

where δ is the Dirac-Delta function.

The principal of linear superposition may be used to define the photon angular flux as the summation of uncollided and collided flux components,
Ψ^{γ}≡Ψ_{unc}^{γ}+Ψ_{coll}^{γ}.
Ψ_{unc}^{γ} is the uncollided, or primary, photon angular flux and Ψ_{coll}^{γ} is the collided, or secondary, photon angular flux. In this context, primary refers to photons which have not yet interacted with the patient anatomy, and secondary refers to photons which were produced or scattered by a photon interaction in the patient anatomy. Substituting Equation (10) into Equation (9) and using linear superposition leads to the following system of transport equations:

$$\begin{matrix}\hat{Ω} \cdot {∇}^{->} {Ψ}_{unc}^{γ} + {σ}_{t}^{γ} {Ψ}_{unc}^{γ} = {q}^{γ} ( E , \hat{Ω} ) δ ( {r}^{->} - {{r}^{->}}_{p} ) , & ( 11 a ) \\ \hat{Ω} \cdot {∇}^{->} {Ψ}_{coll}^{γ} + {σ}_{t}^{γ} {Ψ}_{coll}^{γ} = {q}_{coll}^{γγ} + {q}_{unc}^{γγ} , & ( 11 b ) \\ \hat{Ω} \cdot {∇}^{->} {Ψ}^{e} + {σ}_{t}^{e} {Ψ}^{e} - \frac{\partial }{\partial E} {S}_{R} {Ψ}_{e} = {q}^{ee} + {q}_{coll}^{γ e} + {q}_{unc}^{e} + {q}^{e} , & ( 11 c )\end{matrix}$$

The solution to Equations (11) is identical to that of Equations (9), however, Equation (11a) is decoupled from the other two equations and can be solved independently. Once the solution to Equation (11a) is known, q_{unc}^{γγ} and q_{unc}^{γe} are formulated and considered fixed sources in Equations (11b) and (11c), which only need be solved within the patient anatomy. These sources are generally referred to as the first scattered photon and first scattered electron sources, respectively.

The desired property of Equation (11a) is that Ψ_{unc}^{γ} can be solved for analytically. Doing so provides the following expression for the uncollided photon angular flux from a point source:

$$\begin{matrix}{Ψ}_{unc}^{γ} ( {r}^{->} , E , \hat{Ω} ) = δ ( \hat{Ω} - {\hat{Ω}}_{{r}^{->} , {{r}^{->}}_{p}} ) \frac{{q}^{γ} ( e , \hat{Ω} )}{4 π} \frac{{ⅇ}^{- r ( {r}^{->} , {{r}^{->}}_{p} )}}{{\vert {r}^{->} - {{r}^{->}}_{p} \vert}^{2}} , \\ where , & ( 12 ) \\ {\hat{Ω}}_{{r}^{->} , {{r}^{->}}_{p}} = \frac{{r}^{->} - {{r}^{->}}_{p}}{‖ {r}^{->} - {{r}^{->}}_{p} ‖} , & ( 13 )\end{matrix}$$

and τ(\vec{r},\vec{r}ₚ) is the optical distance (measured in mean-free-paths) between \vec{r} and \vec{r}ₚ, the source and destination points, respectively, of the ray trace.
Adjoint Coupled Photon-Electron Transport Equations

The solution to the adjoint form of Equations (1a) and (1b) represents the contribution that a particle at any location, angle, and energy in the computational domain, will have to the response in the region where the adjoint source is specified. For this reason, the adjoint flux is often time referred to as the importance function. The nature of the adjoint source determines what the adjoint calculated importance field represents. In photon beam radiotherapy dose calculations, the desired response is generally the dose deposition at each location, or dose region, of interest. For a dose region, i, the dose deposition response is given by:

$$R = {D}_{i} = {\int }_{0}^{∞} ⅆ E {\int }_{4 π} ⅆ \hat{Ω} {\int }_{{V}_{vox}^{i}} ⅆ V \frac{{σ}_{ED}^{e}}{ρ} {Ψ}^{e} , \qquad ( 14 )$$

Using the solution to the adjoint operators to Equations (1), one can calculate the above response from any prescribed photon or electron source with variations in space, angle, and energy, provided that the proper adjoint source is specified. This same response for a known volume source would be given by:

$$R = {\int }_{0}^{∞} ⅆ E {\int }_{4 π} ⅆ \hat{Ω} {\int }_{{V}_{src}} ⅆ V [ {q}^{e} {\tilde{Ψ}}^{e} + {q}^{γ} {\tilde{Ψ}}^{γ} ] , \qquad ( 15 )$$

where \tilde{Ψ}^{γ}(\vec{r},E,\hat{Ω}) is the adjoint photon angular flux, \tilde{Ψ}(\vec{r},E,\hat{Ω}) is the adjoint electron angular flux, and q^{γ}(\vec{r},E,\hat{Ω}) and q^{e}(\vec{r},E,\hat{Ω}) are the extraneous photon and electron sources, respectively, defined in volume, V_{src}. For a boundary source, this same response would be given by:

$$R = {\int }_{0}^{∞} ⅆ E {\int }_{\hat{Ω} \cdot {n}^{->} < 0} ⅆ \hat{Ω} {\int }_{δ {V}_{src}} ⅆ δ V \vert \hat{Ω} \cdot {n}^{->} \vert \vert {q}_{surf}^{e} {\tilde{Ψ}}^{e} + {q}_{surf}^{γ} {\tilde{Ψ}}^{ψ} \vert , \qquad ( 16 )$$

where q_{surf}^{γ} (\vec{r},E,\hat{Ω}) and q_{surf}^{e} (\vec{r},E,\hat{Ω}) are the incoming photon and electron sources, respectively, defined on the surface, δV_{src}.

For a spatial domain with volume, V, and surface, δV, the time-independent, three-dimensional system of adjoint Boltzmann transport equations, the exact adjoint of Equations (1), to be solved are given by (for brevity the dependent variables have been suppressed in the equations):

$$\begin{matrix}- \hat{Ω} \cdot {∇}^{->} {\tilde{Ψ}}^{γ} + {σ}_{t}^{γ} {\tilde{Ψ}}^{γ} = {\tilde{q}}^{γγ} + {\tilde{q}}^{e γ} , & ( 17 a ) \\ - \hat{Ω} \cdot {∇}^{->} {\tilde{Ψ}}^{e} + {σ}_{t}^{e} {\tilde{Ψ}}^{e} - {S}_{R} \frac{\partial }{\partial E} {\tilde{Ψ}}^{e} = {\tilde{q}}^{ee} + {\tilde{q}}^{e} , & ( 17 b )\end{matrix}$$

where, as stated earlier, in order for Equations (15) and (16) to be applicable for the dose response, the proper adjoint source must be chosen. For the desired response given in Equation (14), this adjoint source must be defined as:

$${\tilde{q}}^{e} ( {r}^{->} , E , \hat{Ω} ) = \frac{{σ}_{Ed}^{e} ( {r}^{->} , E )}{ρ} , {r}^{->} ∈ {V}_{vox}^{i} . \qquad ( 18 )$$

The adjoint scattering and production sources are defined by:

$$\begin{matrix}{\tilde{q}}^{γγ} ( {r}^{->} , E , \hat{Ω} ) = {\int }_{∞}^{0} ⅆ {E}^{′} {\int }_{4 π} ⅆ {Ω}^{′} {σ}_{s}^{γγ} ( {r}^{->} , E -> {E}^{′} , \hat{Ω} \cdot {\hat{Ω}}^{′} ) {\tilde{Ψ}}^{γ} ( {r}^{->} , {E}^{′} , {\hat{Ω}}^{′} ) , & ( 19 a ) \\ {\tilde{q}}^{ee} ( {r}^{->} , E , \hat{Ω} ) = {\int }_{∞}^{0} ⅆ {E}^{′} {\int }_{4 π} ⅆ {Ω}^{′} {σ}_{s}^{ee} ( {r}^{->} , E -> {E}^{′} , \hat{Ω} \cdot {\hat{Ω}}^{′} ) {\tilde{Ψ}}^{e} ( {r}^{->} , {E}^{′} , {\hat{Ω}}^{′} ) , & ( 19 b ) \\ {\tilde{q}}^{e γ} ( {r}^{->} , E , \hat{Ω} ) = {\int }_{∞}^{0} ⅆ {E}^{′} {\int }_{4 π} ⅆ {Ω}^{′} {σ}_{s}^{γ e} ( {r}^{->} , E -> {E}^{′} , \hat{Ω} \cdot {\hat{Ω}}^{′} ) {\tilde{Ψ}}^{e} ( {r}^{->} , {E}^{′} , {\hat{Ω}}^{′} ) , & ( 19 c )\end{matrix}$$

In general, vacuum boundaries are applied for photon beam radiotherapy applications.
Description of the Calculation Process

This process can be described as being performed in the following steps. (1) First, locations where the dose is desired are specified, referred to as dose regions. For each dose region, steps 2 and 3 are performed. (2) An adjoint electron transport calculation is performed, on a localized computational grid surrounding the dose region, to calculate the adjoint electron flux field, which is stored for future use. This calculation will also compute the adjoint photon source, used as input to step 3. (3) An adjoint photon transport calculation is performed, on a computational grid which may have larger extents than that used for the electron transport, to calculate the adjoint photon flux field, which is stored for future use. (4) Potential beam locations are identified. (5) For each potential beam location, ray tracing is performed to compute the first scattered photon and first scattered electron sources in the patient anatomy, as a function of the prescribed photon energy. (6) The adjoint dose response is computed for each dose region by combining the first scattered source output from step 5, with the adjoint flux solutions calculated in steps 2 and 3. The output of step 6 is the dose in each dose region, resulting from a prescribed photon fluence, at a given beam position, photon direction, and photon energy. (7) Data is transferred to a treatment planning system to be used for patient dose calculations.

There are many variations to the above process. For example, in one embodiment potential beam locations (step 4) and ray tracing (step 5) may be performed prior to the adjoint calculations (steps 2 and 3). In this case, steps 2, 3 and 6 may be repeated sequentially for each dose region, eliminating the need to store the adjoint flux fields for future use.

Step 1: Specification of Dose Regions

Following imaging, a radiation oncologist will generally contour the acquired image to identify regions such as the planning treatment volume (PTV), organs-at-risk (OAR), and other critical structures. Hereafter, contoured structures are referred to as regions of interest (ROI). Additionally, other locations where the dose is of interest, such as individual points, may also be specified. This data may be saved in a format such as DICOM. Treatment plans are often optimized using dose volume histograms (DVHs) of ROIs. In such cases, the dose distribution throughout each ROI is desired, rather than a single dose value integrated over each ROI. In some cases, a single integrated ROI dose may also be of interest. For example, integrated ROI doses may be used for the purposes of optimizing beam directions, or for objectives such as whole body doses.

A grid comprised of 3-D elements is created 601, which may encompass the imaged anatomical region 602. Hereafter, this is referred to as the adjoint computational grid. The process described below assumes that each dose region may be represented by a single element in the adjoint computational grid, or a collection of individual elements. For example, the dose at a point may be represented by a single element in which that point is contained. If only the integrated dose is desired within an ROI, and not a dose distribution, a single dose region may consist of a collection of elements which are contained within, or overlap, that ROI.

If a dose distribution is desired throughout an ROI, for example a PTV 701 or an OAR 702, the ROI may be represented by a collection of individual elements 703, with each element is defined as a separate dose region. In this manner, the number of dose regions may be equivalent to the number of elements which are contained within or overlap that ROI. Alternatively, if a dose distribution is desired throughout the entire patient volume, the number of dose regions may be equivalent to the number of elements which are contained within or overlap the relevant patient volume 601.

To summarize the above, a single element will generally equate to a single dose region, except when only an integrated dose is desired throughout a region, and not a dose distribution, or in regions where a fine spatial resolution is not required. In such cases, a collection of elements may comprise a single dose region. The list of dose regions, i , and the elements associated with each dose region, is stored in computer memory or on disk.

Material properties from the acquired image data may be mapped to each element in the primary computational grid, using a prescribed relationship to convert CT Hounsfield numbers, or other image data, to a material type (bone, tissue, adipose tissue, etc.) and density. Many methods exist for this process.

Many other embodiments exist. For example, the adjoint computational grid may be comprised of 3-D element types other than Cartesian elements, such as variably sized, arbitrarily shaped tetrahedral or polyhedral elements, or combinations of element types. In one embodiment, these elements may be body fitted to ROI surfaces. Alternatively, separate adjoint computational grids may be constructed for each dose region. For example, a separate polar grid may be used for each dose region, where the dose region is located in the central element(s), and a radially variable grid spacing is employed.

Step 2: Adjoint Electron Transport Calculation

In the current description, steps 2 and 3 are performed for a single dose region, i, then are repeated iₜₒₜ times, where iₜₒₜ is the total number of dose regions.

A computational grid is constructed which includes the dose region element 801 and neighboring elements 802 out to a prescribed distance. Hereafter, this computational grid is referred to as the adjoint electron grid. In one embodiment, the adjoint electron grid may be created as a subset of the adjoint computational grid 803, where all elements in the adjoint electron grid are also contained in the adjoint computational grid.

The adjoint electron grid extents are generally created such that the distance from the dose region to the grid perimeter is large enough so that the contribution of electrons beyond the grid perimeter would be insignificant to the dose in the dose region.

An isotropic volumetric adjoint electron source, \tilde{q}^{e}, defined in Equation (18), is applied to the dose region. If dose-to-medium is desired, σ_{ED}^{e} may be based on the material of the adjoint source region. If dose-to-water is desired, σ_{ED}^{e} may be based on water. Other embodiments exist.

A solver which calculates the solution to Equation (17b), the first of the two coupled adjoint equations, is then called. Input to this solver includes the adjoint electron grid, with material properties associated with each grid element, and the adjoint electron source in the dose region. The solver will calculate the adjoint electron flux, \tilde{Ψ}^{e}, everywhere in the adjoint electron grid, which is used as input to step 6. A second output of this calculation is the adjoint photon source, \tilde{q}^{eγ}, defined by Equation (19c) and used as input to step 3. \tilde{Ψ}^{e} is then stored on a grid of voxels referred to as the response grid.

The response grid may consist of voxels encompassing the patient anatomy. In one embodiment, the adjoint electron grid may be a subset of the response grid, where voxels in the response grid are of identical shapes and sizes to elements in the adjoint electron grid.

Many other embodiments exist for this process. Many different deterministic solution methods could be used to calculate the solution to Equation (17b), which may be known by those skilled in the art. Alternatively, \tilde{Ψ}^{e} and \tilde{q}^{eγ} could be calculated using an adjoint Monte Carlo method, or using analytic or empirical methods. In one embodiment, a deterministic solution method using linear or higher order finite element spatial differencing is employed. In this manner, larger adjoint electron grid elements may be used than the voxels in the response grid, and \tilde{Ψ}^{e} may be mapped to the response grid voxels based on the finite element spatial solution representation calculated within each adjoint electron grid element.

The adjoint solution field data calculated in step 2 may be stored, perhaps in computer memory or on disk, for use in steps 3 and 6. There are many data compression techniques which may be used to reduce the data storage requirements, which are too numerous to mention herein.

Step 3: Adjoint Photon Transport Calculation

An adjoint photon grid 901 is created, which includes all computational elements of the electron transport grid 902 and additional surrounding elements. The optical distance to the grid perimeter is generally selected to be large enough so that the dose contribution of photons beyond the grid perimeter would be insignificant to the dose in the dose region.

The adjoint photon source, \tilde{q}^{eγ}, calculated in step 2 for every element in the adjoint electron grid, is mapped to corresponding elements of the adjoint photon grid.

A deterministic solver which calculates the solution to Equation (17a) is then called. Input to this solver includes the adjoint photon grid with material properties associated with each grid element, and the adjoint photon source.

The solver will calculate the adjoint photon flux, \tilde{Ψ}^{γ}, everywhere in the adjoint photon grid, along with the adjoint electron flux calculated in step 2, the adjoint photon flux is used as input to step 6. \tilde{Ψ}^{γ} is then stored on corresponding voxels of the response grid.

Similar to step 2, many embodiments exist for this process. First, there are multiple deterministic solution methods which could be used to calculate the solution to Equation (17a), which are known to those skilled in the art. Alternatively, \tilde{Ψ}^{γ} could be calculated using an adjoint Monte Carlo method, or using analytic or empirical methods. In one embodiment, a deterministic solution method using linear or higher order finite element spatial differencing is employed. In this manner, larger adjoint electron grid elements may be used than the voxels in the response grid, and \tilde{Ψ}^{γ} may be mapped to the response grid voxels based on the finite element spatial solution representation calculated within each adjoint photon grid element.

In another embodiment, the adjoint photon grid may be separate from the adjoint electron grid, with different element sizes and shapes. In this embodiment, the adjoint photon source field, computed in step 3, may be mapped over to corresponding elements of the adjoint photon grid.

The adjoint solution field data calculated in step 3 may be stored, perhaps in computer memory or on disk, for use in step 6. There are many data compression techniques which may be used to reduce the data storage requirements, which are too numerous to mention herein.

Step 4: Specification of Potential Beam Directions

In photon beam radiotherapy, beams are delivered to the patient from multiple directions. In this context, beam direction refers to the position and orientation of the gantry, or treatment head, associated with a delivered beam. In general, beam directions are not specified prior to treatment planning, as beam directions may also be optimized during the treatment planning process.

For each beam direction, the radiation source incident on the patient may be represented through a variety of methods. In one embodiment, the photon source may be modeled by a single anisotropic focal point source, located at the treatment head target for that beam direction. In this case, all photons incident on the patient are assumed to be traveling in the focal direction, defined by rays originating at the point source location. In another embodiment, the photon source may be modeled using a point source at the target, and a second point source located at the flattening filter, to represent extra-focal scatter. Other embodiments exist. For simplicity in the current description, the photon source from each beam direction is assumed to originate solely from a single point source. However, the addition of multiple point sources per beam direction needs a straightforward duplication of the identical process described for a single point source, but for a different point source location and anisotropy description.

For each point source, n, the source location and orientation may be defined by an origin point and beam direction vector, which is parallel to the centerline of the beam originating from that source. The source anisotropy may be described by a fluence map, oriented normally to the beam direction vector. In this context, photon fluence, ψ^{γ}, is referring to the time integrated photon source intensity over a specified time, t₁, which is related to the photon flux as follows:

$${ψ}^{γ} = {\int }_{t = 0}^{{t}_{1}} {Ψ}^{γ} ⅆ t \qquad ( 20 )$$

At each fluence map grid element, defined by a unique position (k,l) on the fluence map grid, ψ^{γ} may be independently prescribed for each photon energy group, g. Thus, the total ψ^{γ} array size is (N×K×L×G), where: N is the number of specified point sources; K and L are the number grid elements in the k and l directions of the fluence maps, respectively; and G is the number of photon energy groups used to describe the prescribed photon fluence. In one embodiment, K, L, and G may be different for each point source.

For a given dose region, i, the output of step 6 is the dose response, Rᵢ, for a prescribed photon fluence of ψ^{γ}(n,k,l,g). Here, ψ^{γ}(n,k,l,g) may represent q^{γ}(E,\hat{Ω}) for a given source position, \vec{r}ₚ, in Equation (9a). The total dose in i from a prescribed treatment is therefore the summation of the dose responses from the prescribed photon fluence in each fluence map grid element, for all photon energies and source positions:

$${D}_{i} = {\sum }_{n = 1}^{N} {\sum }_{k = 1}^{K} {\sum }_{l = 1}^{L} {\sum }_{g = 1}^{G} {R}_{i} ( {ψ}^{γ} ( n , k , l , g ) ) \qquad ( 21 )$$

Since the calculation speed of steps 2 through 6 is largely independent of the fluence map spatial resolution, a finer spatial resolution fluence map may be used than is needed by the treatment planning system during optimization.

Many other embodiments exist. For example, in SRS a finite number of collimated beam shapes may exist, where angular variations in the photon intensity and spectrum for each collimator are fixed, and only the total fluence may be modified during treatment plan optimization. In such cases, the fluence map may be described by a fixed radial distribution of the photon fluence for each energy group. Following completion of step 6, this information may be compressed such that the dose response is saved only as a function of the total fluence for that collimator position, since the radial distribution is known and fixed.

The first scattered photon and electron sources calculated in step 4 may be stored, perhaps in computer memory or on disk, for use in step 6. There are many data compression techniques which may be used to reduce the data storage requirements, which are too numerous to mention herein.

Step 5: Ray Tracing to Compute the First Scattered Photon and Electron Sources

In this step, ray tracing 1001 is performed from each point source 1002 to compute the first scattered photon sources, q_{unc}^{γγ}, and first scattered electron sources, q_{unc}^{γe}, in the patient anatomy 1003. In one embodiment, these will be computed on the response grid voxels. Material properties from the acquired image data may be mapped to each voxel, using a prescribed relationship to convert CT Hounsfield numbers, or other image data, to a material type (bone, tissue, adipose tissue, etc.) and density. Ray tracing is performed using the process described in Equation (12), which can be used to compute Ψ_{unc}^{γ} at each voxel. By using the material cross sections in each voxel, q_{unc}^{γγ} and q_{unc}^{γe} are then calculated through Equations (3a) and (3b), respectively. These sources may be represented as spherical harmonics moments, as given by Equation (7).

Each ray trace will pass through a (k,l) grid element location on the associated fluence map 1004. Ray traces only need to be performed for angles which intersect the fluence map.

For each voxel to which ray tracing was performed, information saved, either in memory or on disk, includes q_{unc}^{γγ}(n,k,l,g) and q_{unc}^{γe}(n,k,l,g).

Both q_{unc}^{γγ} and q_{unc}^{γe} are calculated separately for each incident photon energy group, g, and both sources may be described using a energy group structure independent of the incident photon source energy group structure.

For simplicity, the current description assumes that both the response grids and the adjoint calculation grids are identical, with the exception that the adjoint calculation grids may represent a subset of the response grid. However, many embodiments of the above process may exist. In one embodiment, the response grid may consist of variably sized and shaped elements. In another embodiment, different response grids may be associated with different source regions. In another embodiment, τ(\vec{r},\vec{r}ₚ) may be calculated using a different spatial resolution than the response grid voxels where q_{unc}^{γγ} and q_{unc}^{γe} are calculated. Other ray tracing methods may also be applied. Many other embodiments exist.

Step 6: Calculation of the Dose Response

The outputs from steps 2 and 3 are the adjoint electron flux, \tilde{Ψ}^{e}, and the adjoint photon flux, \tilde{Ψ}^{γ}, respectively, at voxels in the response grid, V. The outputs from step 5 are the first scattered photon source, q_{unc}^{γγ}, and first scattered electron source, q_{unc}^{γe}, at voxels in the response grid, and computed for each dose region, i. These are used to construct the dose, Dᵢ, in dose voxel, i, from a prescribed photon fluence, ψ^{γ}(n,k,l,g), as follows:

$${D}_{i} ( n , k , l , g ) = {\int }_{0}^{∞} ⅆ E {\int }_{4 π} ⅆ \hat{Ω} {\int }_{V} ⅆ V [ {q}_{unc}^{γ e} {\tilde{Ψ}}^{e} + {q}_{unc}^{γγ} {\tilde{Ψ}}^{γ} ] + {\int }_{0}^{∞} ⅆ E {\int }_{4 π} ⅆ \hat{Ω} {\int }_{{V}_{i}} ⅆ V \frac{{σ}_{ED}^{γ}}{ρ} {Ψ}_{unc}^{γ} \qquad ( 22 )$$

Here,

$${\int }_{V} ⅆ V$$

represents the volume integration over all voxels in the response grid,

$${\int }_{{V}_{i}} ⅆ V$$

represents the volume integration over the dose voxel,

$$i , {\int }_{4 π} ⅆ \hat{Ω}$$

represents the angular integration over all angles, and

$${\int }_{0}^{∞} ⅆ E$$

represents the energy integration over all energy groups for which the adjoint flux and first scattered sources are computed.

The second term on the right of Equation (22) represents the direct dose deposition from primary photon interactions. However, since the energy deposition photon cross section, σ_{ED}^{γ}, is small, this contribution can generally be neglected in radiotherapy.

The total dose resulting from a given treatment plan, is given by Equation (21), which represents the summation of Equation (22) over all fluence map grid elements for all source positions and all prescribed photon energy groups.

The response data calculated in step 6 may be stored, perhaps in computer memory or on disk, for use in step 7. There are many data compression techniques which may be used to reduce the data storage requirements, which are too numerous to mention herein.

Step 7: Interfacing with a Treatment Planning System

Steps 1 through 6 may be performed prior to treatment planning, and need only the acquired image data, a list of potential beam positions, and optionally, ROIs. Such data may optionally be exported from a treatment planning system in a standardized format such as DICOM-RT. The process of performing steps 1 through 6 for thousands of adjoint source locations is computationally intensive, but can be performed independently of a treatment planning system. Thus, this process may be performed on a dedicated multi-processor workstation, after the radiation oncologist has contoured the image data, and prior to treatment planning begins.

During treatment planning, this data may be read into a treatment planning system, perhaps for only a subset of the source positions calculated.

Accounting for Motion

In the course of a radiotherapy treatment, the patient anatomy may considerably vary between treatment fractions, or even within a single fraction. In such cases, the above process may be adapted to account for anatomical motion through the following steps. First, steps 2 and 3 are performed for each dose region, using the original acquired image data, to compute and store \tilde{Ψ}^{e} and \tilde{Ψ}^{γ} for each dose region. A second image may be acquired at the time of treatment, and through a registration process, the response grid elements on which \tilde{Ψ}^{e} and \tilde{Ψ}^{γ} are calculated, may be mapped to locations on the newly acquired image data. A variety of registration techniques exist, which are well known to those skilled the art. Steps 4 and 5 may then be performed on the newly acquired image data to calculate q_{unc}^{γγ} and q_{unc}^{γγ} from which step 6 is performed.

If motion changes are substantial, corrections may be applied to \tilde{Ψ}^{e} and \tilde{Ψ}^{γ}. A simple example of this is to introduce a correction based on the distance between a response grid voxel, j, and a dose region, i. For example, if the distance between i and j on the original acquired image is r₁, and the distance between i and j after registration on the new image is r₂, \tilde{Ψ}^{e} and \tilde{Ψ}^{γ} may be multiplied by a factor of (r₁/r₂)² to obtain corrected values on the new image data. Numerous other embodiments exist.

Alternative Embodiments

Some alternative embodiments of the above process are described below. There are many others which are too numerous to describe.

In one embodiment, the above process may be used to determine optimal beam angles. In this embodiment, a single dose region may be prescribed in each ROI, which encompasses the volume of each ROI.

There are many embodiments in which the ray tracing and adjoint solutions may be combined. In one embodiment, the ray tracing may be performed using integral transport theory to calculate the adjoint photon angular flux at a prescribed point source location, \vec{r}ₚ, external to the anatomical region. In this embodiment, steps 2 and 3 solve for \vec{q}^{eγ} and \tilde{q}^{γγ}, respectively, within the anatomical region, and integral transport theory may be used to solve for \tilde{Ψ}^{γ}(\vec{r}ₚ,E,\hat{Ω}_{\vec{r},\vec{r}}_{ₚ}). Equation (22) for Dᵢ becomes:

$$\begin{matrix}{D}_{i} = {\int }_{0}^{∞} ⅆ E {\int }_{V} ⅆ V {q}^{γ} ( E , {\hat{Ω}}_{\vec{r} , {\vec{r}}_{p}} ) [ {\tilde{q}}^{e γ} ( \vec{r} , E , - {\hat{Ω}}_{\vec{r} , {\vec{r}}_{p}} ) + \\ {\tilde{q}}^{γ γ} ( \vec{r} , E , - {\hat{Ω}}_{\vec{r} , {\vec{r}}_{p}} ) ] \frac{{ⅇ}^{- τ ( \vec{r} , {\vec{r}}_{p} )}}{{\vert \vec{r} - {\vec{r}}_{p} \vert}^{2}}\end{matrix} \qquad ( 23 )$$

Other embodiments exist.

In one embodiment, a KERMA approximation may be employed for electrons, which may be selectively applied for specific dose regions, for example those outside critical structures. In this embodiment, Equation (22) becomes:

$$\begin{matrix}{D}_{i} = {\int }_{0}^{∞} ⅆ E {\int }_{4 π} ⅆ \hat{Ω} {\int }_{V} ⅆ V {q}_{unc}^{γ γ} \tilde{{Ψ}^{γ}} + {\int }_{0}^{∞} ⅆ E {\int }_{4 π} ⅆ \hat{Ω} {\int }_{{V}_{i}} ⅆ V \\ \frac{{σ}_{KERMA}^{γ} ( E , \vec{r} )}{ρ} {Ψ}_{unc}^{γ}\end{matrix} \qquad ( 24 )$$

In this embodiment, an electron adjoint calculation (step 2) is not required, and input to the adjoint photon calculation (step 3) may be an isotropic adjoint photon source in the dose region, i, defined by:

$${\tilde{q}}^{γ} ( \vec{r} , E , \hat{Ω} ) = \frac{{σ}_{kerma}^{γ} ( \vec{r} , E )}{ρ} , \vec{r} ∈ {V}_{i} \qquad ( 25 )$$

where σₖₑᵣₘₐ^{γ} may be the KERMA cross section for the material of dose region i, or alternatively, if dose-to-water is desired, the KERMA cross section may be based on water. Other embodiments exist. Similar to the process in step 3, an adjoint photon grid may be constructed surrounding the dose region, and the adjoint photon transport calculation is performed to compute \tilde{Ψ}^{γ}. Many embodiments exist when using the KERMA approximation. In one embodiment, a KERMA approximation may only be applied to electrons produced by secondary photon interactions.

Owing to relatively large mean free paths of photons, the gradients in the dose field produced by secondary photons are relatively gradual. As a result, when a KERMA approximation is employed, larger dose regions may be used in the adjoint photon transport calculations than are used for the adjoint electron transport calculations. In one embodiment, \tilde{Ψ}^{γ} may also be stored on a response grid of larger voxels than is used to store \tilde{Ψ}^{e}. Numerous other embodiments exist.

When the KERMA approximation is applied, the adjoint photon source in the dose region may be represented as an isotropic adjoint point source, and the adjoint form of Equation (12) may be used to calculate the adjoint uncollided photon flux and the adjoint first scattered distributed photon sources. In one embodiment, an adjoint first scattered distributed point source approach, adapted for charged particles, may also be applied to represent the adjoint electron sources. Other embodiments may exist.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the inventions be defined by the claims herein and their equivalents.

1. A method for calculating dose responses in a volume comprising:
specificying of one or more discrete regions where the dose is desired; and
for each dose region,
performing an adjoint calculation to calculate the adjoint solution fields from that dose region, and
performing a ray tracing process to transport the adjoint solution fields out of the volume and to locations where an incident particle fluence may be prescribed.
